(19) United States
(12) Patent Application Publication
(10) Pub. No.: US 2007/0174925 A1
(43) Pub. Date: Jul. 26, 2007

(54) Treatment models and uses thereof

(76) Inventors: Ruth Barrett, Cambridgeshire (GB); Paul Goldsmith, Cambridgeshire (GB); Angeleen Fleming, Cambridgeshire (GB)

Correspondence Address: KLARQUIST SPARKMAN, LLP, 121 SW SALMON STREET, SUITE 1600, PORTLAND, OR, 97204, US

(21) Appl. No.: 10/563,628

(22) PCT Filed: Jul. 7, 2004
(86) PCT No.: PCT/GB04/02922

§ 371 Date: Feb. 15, 2007

(30) Foreign Application Priority Data
Jul. 8, 2003 (GB) 0315995.1

(51) Int. Cl. A01K 67/027

(52) U.S. Cl. 800/20

(57) ABSTRACT

The generation and use of disease models in fish such as zebrafish are provided, in particular use in a screen to identify a gene or test substance that affects a disease state associated with pain or nociception, or pain or nociception. In particular, fish models are used in a screen to identify a gene or test substance that affects activity or effect of a second gene provided transgenically that affects a pain or nociception state of the fish, or a treatment of the fish that affects a pain or nociception state of the fish. A pain state or nociception of model fish is compared with that of mutated or treated fish, allowing for identification of genes and test substances with therapeutic application.

The present invention relates to generation and use of fish models, for instance in assays to identify and investigate genes and substances involved in disease and disease treatment, identification and use of drug targets. In particular, the present invention relates to generation and use of disease models in fish such as zebrafish.

Reference is made herein to “disease” in various contexts. In relation to the present invention, disease is generally used to refer to disease associated with pain or nociception, or to pain or nociception itself. The invention is applicable to nociception and possibly pain experienced by wild-type fish on application of a stimulus, e.g. excess heat or cold, electric shock. The invention is also applicable to nociception and possibly pain associated with a physical disease or disorder.

The invention particularly relates to use of model fish in screening for and identifying analgesics, and candidate substances for use in pain relief or treatment in other organisms, e.g. humans.

In some preferred embodiments, gradable phenotypes are generated in fish, allowing for degree of correction or alteration of an activity or effect of a treatment, gene or mutation to be assayed.

In some embodiments of the invention, one or more stimuli are applied in order to differentiate degrees of nociception or pain in different fish. For example, a painful stimulus may be applied such as high or low temperatures, electric shocks or adverse compounds. Many such stimuli can be graded. In some preferred embodiments, a competing stimulus can be applied to distinguish the level of nociception, for example a threatening shape (e.g. model of a predatory animal) or varying degrees of shade over the water. Other preferred embodiments are disclosed herein.

This application outlines methods of inducing and rescuing nociception or pain in the zebrafish, and an assay with which the level of nociception can be measured. Together, these tools will allow the discovery of new pain-killing compounds.

Although pain is clearly a protective evolutionary adaptation, there are many situations where it is maladaptive and pathological. Pain perception in humans is a complex phenomenon that depends on multiple parameters, and many patients never find satisfactory solution for their pain. Research into pain with a view to discovering new analgesics has focused thus far on rodent models. A variety of assays exist whereby a pain state is induced in the rodent, the level of which can be monitored. Assays are targeted towards thermal, mechanical or chemical noxious stimuli. Rodent assays are costly to set up and perform, can only be done with relatively small numbers of animals (which therefore often have to be repeatedly tested) and results reiteratively have been impossible to repeat. The variability in results obtained from mammalian assays has made obtaining statistically significant results difficult. There are also ethical issues with assaying pain in mammals.

It would therefore be highly desirable if a method could be identified for the measurement of pain responses in a non-mammalian vertebrate in a reliable and robust fashion, with the capacity to obtain statistically significant results. The present inventors hypothesized that larval fish could be used to achieve these goals.

There has been considerable debate in the literature as to whether fish can experience pain at all, let alone larval fish. It has been argued that since fish do not possess the well developed brain areas involved in the processing of pain signals in man and higher primates that it is unlikely that they feel pain (Bermond et al, 1997). Consciousness may be required for the perception of pain, and it is unclear whether fish are conscious (Rose, 2003).

In contrast, other research suggests that at least some strains of fish do display some nociceptive responses. At the cellular level, trout have recently been shown to have nociceptors that bear many similarities to those of humans (Sneddon et al, 2003, Sneddon 2003). At the behavioral level, experiments have shown that fish learn to avoid noxious stimuli (Beukema, 1970). They react differently to different levels of stimuli presented in a constant environment which has been argued to show that processing of nociceptive signals goes above more than a mere reflex; the escape reflex was not the same in each situation, and a level of conscious experience may play a role in modulating this (Verheigen and Buwalda, 1988). Furthermore, goldfish have been shown to perform in a pain assay typically used in rodents in a similar way to a rat (Ehrensing et al, 1982).

Research into behavioral responses resulting from nociception in fish has thus far focused on fully developed adult fish. Given the debate on the presence or absence of pain responses in adult fish, it is even more unclear as to the presence or absence of pain responses in larval fish.

An additional problem is that, even if a fish could experience pain, how it can be assayed in a sensitive and scalable fashion. This requirement is essential if they are to be useful for screening purposes. The methodology used to date has involved small sample sizes and are similar to rodent experiments in that each animal is tested individually.

The inventors have invented methods of sensitive and scalable assaying for pain responses in fish, both immature larval forms and adult. The methods outlined here permit the use of large sample sizes, with the potential for many individual fish to be tested simultaneously. The assays are quick to perform and require no restraint of the fish.

### BRIEF DESCRIPTION OF THE FIGURES

FIG. 1 illustrates a side view of temperature gradient apparatus useful in assays of the invention.

FIG. 2 illustrates a top view of a temperature gradient setup.

FIG. 3 shows results of a control experiment.

FIG. 4 shows that fish treated with an opiate are more often found in water of a higher temperature than control fish tested simultaneously. t=10 mins.

FIG. 5 shows that fish treated with an endogenous cannabinoid also show less aversion to higher temperatures. t=10 mins.

FIG. 6 shows results indicating that fish sensitized with DNCB choose cooler temperatures very strongly and quickly as they over-react to the temperature. t=5 mins.

FIG. 7 juxtaposes the same data from sensitized fish from FIG. 6 with the same fish treated with an opiate and retested. t=5 mins.

FIG. 8 illustrates a top view of an alternative temperature gradient setup.

FIG. 9 is a line graph representation of the data shown in FIG. 3.

FIG. 10 is a line graph representation of the data shown in FIG. 4.

FIG. 11 is a line graph representation of the data shown in FIG. 5.

FIG. 12 is a line graph representation combining the data shown in FIGS. 6 and 7. FIG. 12 shows results that demonstrate that fish sensitized with DNCB locate to cooler temperatures than unsensitized controls and do so very rapidly. When these same fish are treated with an opiate and retested they lose this sensitivity, and their distribution becomes randomized in the gradient. The controls for these 2 tests are shown for comparison. Assay performed at t=5 mins.

FIG. 13 indicates the degree of hyperalgesia or analgesia (that is, length and direction of histogram bars with respect to the control response). This is derived from the difference in distribution of the fish in the channels between the treated group and the control group for that experiment. The p values are calculated using t tests.

FIG. 14 shows results indicating that three week old fish respond in essentially the same way as younger fish (all other data shown in other figures is obtained using fish less than 10 days old) when treated with an opiate. Opiate treated fish locate to regions of a higher temperature than untreated fish.

Drug discovery is currently limited by the ability to know whether inhibition of a particular gene, biological pathway or a combination of several genes/pathways will have a desirable effect on a particular disease state in vivo. The present invention provides strategies to overcome these problems, including methods to develop the appropriate disease models, how to subsequently screen these models, then how to translate this into human therapeutics. By combining the appropriate steps in a particular way the overall aim is achieved. The invention thus lies not only in the nature of particular individual steps, but also in the particular way these individual steps are combined together. The nature of certain steps places constraints on other steps. Thus, an additional part of the invention lies in the recognition of these constraints and the application of particular strategies, both in that particular step, and in the other steps, with the aim of overcoming these constraints.

The optimal model system for the in vivo practice of the enclosed disclosure is a fish, especially a zebrafish. The zebrafish is an organism which combines many of the advantages of mammalian and invertebrate model systems. It is a vertebrate and thus more relevant to models of human disease than Drosophila or other invertebrates, but unlike other vertebrate models it can readily be used to perform genetic screens.

The inventors have appreciated that zebrafish offer the unique combination of invertebrate scalability and vertebrate modeling capabilities. They develop rapidly, with the basic body plan already having been laid out within 24 hours of fertilization. Moreover, their ex-utero development within a transparent capsule allows the easy in vivo visualization of internal organs through a dissecting microscope. Many disease states can be modeled within the first week of life, at which time the embryos are only a few millimetres long and capable of living in 100 ul of fluid. This permits analysis of individual embryos in multi-channel format, such as 96 well plate format. This is particularly useful for drug screening, with many chemicals being arranged in 96 well plate format.

Alternatively, a population of fish in a petri dish or a tank may be employed. A population of fish may be treated together, and may be tested together, e.g. via addition of one or more or a combination of test substances to the water.

The zebrafish has a short maturation period of two to three months and is highly fecund, with a single pair of adults capable of producing 100 to 200 offspring per week. Both embryos and adults are small, embryos being a few mm and adults 2-3 cm long. They are cheap and easy to maintain. The ability to generate large numbers of offspring in a small place offers the potential of large scalability.

A further advantage of zebrafish is the fact they live in water. This makes administration of candidate chemicals easy. Moreover, the inventors have discovered that zebrafish are also DMSO tolerant. This is important as many drugs are dissolved in DMSO. The inventors have established that zebrafish can tolerate 1% DMSO. Thus, a candidate drug or other test substance may be dissolved in DMSO and administered to zebrafish by adding to the fish water to give a final concentration of DMSO of at least up to 1%. This is employed in various preferred aspects and embodiments of the present invention.

Zebrafish and other fish also readily absorb chemicals. The effective concentration of chemicals in the water seems to equate to the effective plasma concentration in mammals.

Thus, zebrafish enable the entire biological pathway of a vertebrate to be screened in a high-throughput fashion.

It is possible to introduce random mutations into the zebrafish genome, for example with the use of chemical mutagenesis (Solnica-Krezel et al., Genetics 1994, 136(4): 1401-20).The publication of the results of the first large scale mutagenesis screens was in 1996 by the Nüsslein-Volhard and Driever groups [Driever, 1996; Haffter, 1996]. They were able to isolate over 2000 mutants affecting nearly every aspect of embryogenesis during the first few days of development. The original 1996 screens analyzed 2746 strains of embryos. Only those with obvious morphological abnormalities visible under the dissecting microscope were kept. Thus the fish selected on the basis that they might have a visual problem were those with alterations in eye shape, size or pigmentation. 49 mutants were isolated this way. Such screening methods inevitably introduce bias into mutant selection. It is also likely that only a small fraction of the total number of possible mutants were identified, as most of the loci isolated were only represented by a single allele.

Various strategies have been devised to increase the sensitivity of primary mutagenesis screens. In the original screens, microscopic examination was used to detect degeneration of particular anatomical areas of interest, by observing a decrease in the size of the structure or altered optical properties using Nomarski optics. Now, groups are creating transgenic lines expressing GFP under the control of promoters, thus expressed in subtypes of cells of interest to speed up screens, similar to that previously used in other animals (e.g. W001/12667). Mutant fish can then be screened for loss of fluorescence and thus abnormal development or degeneration of a particular cell. An example is the transgenic line expressing GFP in rod photoreceptors, by placing the rod opsin promoter upstream of eGFP (Kennedy et al., J Biol Chem (2001) 276, 14037-14043). It is also possible to make transgenic fish carrying exogenous genes. Zebrafish expressing a heterologous Ikaros protein have been used to model haematopoiesis and lymphoproliferative disorders (WO 01/40273).

To add sensitivity to the assessment of the degree of pain, a non-behavioral marker may be measured. For example, it is known that certain genes are differentially expressed in particular pain conditions. For example, certain sodium channels may be up or down regulated in the dorsal route ganglia with a chronic pain state. Additionally, genes such as the immediate early genes, for example f-fos, may be activated during a painful state in those subsets of neurons responsible for subserving the painful stimuli. By measuring one of these markers, it is possible to avoid the need for the measurement of a purely behavioral output, increasing the scope and sensitivity of the pain assay.

Methods by which this may be achieved include the use of RT-PCR, in situ hybridizations and immunohistochemistry. A further elegant way involves the generation of a transgenic zebrafish in which the promoter of a gene differentially expressed in a pain state is coupled to a marker such as a fluorescent protein. Thus, for example, when the fish is exposed to a painful stimuli an increase in fluorescence may be seen. An analgesic may therefore be detected by the alteration of this response.

A number of studies have already been published analyzing neuronal activation in zebrafish. All such studies have used calcium as a measure of neuronal activity, either using calcium sensitive reporter dyes (Fetcho et al., 1998; Zimprich et al., 1998; Ashworth and Bolsover, 2002), or using transgenic lines (Higashijima et al., 2003). There are a variety of methods that can be employed to look at neuronal activation in zebrafish, as described below.

Altered c-fos expression can be assessed by immunohistochemistry. c-fos expression is upregulated in a tissue/region-specific manner in response to a variety of stimuli e.g. pain, addiction, wounding. Antibodies are available that cross-react with zebrafish c-fos and other groups have already demonstrated that c-fos activation at wound sites can be detected using such antibodies (P. Martin, personal communication). We have successfully used this approach to look for changes in c-fos expression in specific brain regions in our addiction model and would predict that similar changes can be observed following exposure to noxious stimuli. Using fluorescent secondary antibodies, we can measure fluorescent intensity and hence detect increases or decreases in c-fos expression.

Calcium imaging can be carried out by injection of tracers. A variety of calcium sensitive dyes are available, many of which have been used in zebrafish to look at calcium transients during development (Creton et al., 1998; Webb and Miller, 2003) and neuronal activation (Fetcho et al., 1998; Zimprich et al., 1998; Ashworth and Bolsover, 2002). Such dyes can be injected at the blastocyst stage, and hence become incorporated into most/all cells. Alternatively, neurons can be labelled by retrograde filling. The strength of this approach is that it allows the imaging of neuronal activation in the living animal.

c-fos activation can be studied in transgenic reporter lines. Generating zebrafish transgenic lines is a relatively straight-forward process, and one that the authors are very experienced in. Development of transgenic reporter lines, for example, using destabilized GFP (or similar reporter) under the control of the c-fos promoter would allow high throughput analysis of changes in neuronal activation following exposure to noxious stimuli.

Calcium imaging can be carried out in transgenic reporter lines. Cameleon is a genetically encoded calcium indicator which has been used to generate transgenic reporter lines in C. elegans and zebrafish (Kerr et al., 2000; Higashijima et al., 2003). Cameleon is a hybrid protein in which CFP and YFP are linked by calmodulin and an M13 calmodulin binding domain. Following an increase in calcium levels, calmodulin binds calcium and interacts with M13 causing a conformational change in the protein. This change results in a transfer of fluorescence resonance energy from CFP to YFP. Ratiometric measurements of CFP/YFP can therefore be used as a ratiometric calcium indicator and hence a measure of neuronal firing.

WO99/42606 concerns a method of screening an agent for an angiogenesis activity or cell death activity or toxic activity, comprising administering the agent to a teleost (e.g. zebrafish, medaka, Giant rerio or puffer fish), and detecting a response in the teleost indicating angiogenesis activity or an effect on cell death activity or toxic activity in at least one tissue or organ of the teleost.

WO01/12667 describes use of a transgene to drive marker expression in the eye. The organism may be fish. It suggests making a transgenic animal (which it says may be a fish) by a method comprising introducing a genetic construct for expression of a marker sufficient to visually detect the marker in photoreceptive cells or organ and selecting for transgenesis by visually detecting the marker in a photoreceptive cell or organ.

WO01/51604 (Exelixis) is concerned with providing sensitizer genes such as a tumor gene or an oncogene in a non-human animal (for which zebrafish are mentioned as a passing, hypothetical possibility) in cells where expression is non-lethal. It is proposed to detect changes and compare on mutation or other treatment. The aim is identification of “interactor genes” that, when mutated, specifically kill or reduce the size of target tissue (subject to the sensitizer gene).

WO98/56902 discloses use of transgenic fish, including zebrafish, and methods of crossing fish strains, including strains with mutations, the aim being to identify genes that affect expression of fish genes.

Scott C. Baraban, Peter A. Castro and Herwig Baier have disclosed identification of seizure resistant zebrafish mutants as a model of epilepsy. For induction of seizures, zebrafish larvae were exposed to a common proconvulsant agent (pentylenetetrazole, PTZ) and the fish were observed to undergo three distinct stages of seizure-like behavior, as described previously (Baraban et al. 2001; Epilepsia 42:3.017). Twelve families that carried putative seizure-resistance mutations were identified.

In contrast to the above disclosures, the present invention provides particular methods which enable a fish, such as a zebrafish, to be used efficiently in a wide variety of situations to discover therapeutics relevant to human disease. Provided by the present invention are disease modeling methods which are also particularly amenable for use in screening. This allows in turn to the identification of a human therapeutic. The invention is specifically concerned with treatment of pain, screening for and identifying analgesic substances.

The present invention provides means, specifically fish such as zebrafish, for use in inventive methods of screening for and identifying a gene which, when mutated, alters the activity or effect of a second gene, involved in pain transduction, and thus perception of pain. A secondary gene of which mutation affects activity or effect of a primary gene is termed an “interactor” gene and if it reduces activity or effect of a primary gene is termed a “suppressor” gene. If it increases activity of effect of a primary gene it is termed an “enhancer” gene. Interactor genes, including suppressor and enhancer genes, represent targets for drugs to treat pain. A particular advantage of interactor gene screens is that the interactor gene may be part of an unexpected pathway, but will still be identified in such a screen. Additionally, as drugs commonly bind to and antagonize their targets, a drug which binds to the protein encoded by the wild type interactor gene, may have a similar beneficial effect on pain.

In addition to zebrafish, other fish such as fugu, goldfish, medaka and giant rerio are amenable to manipulation, mutation and study, and use in aspects and embodiments of the present invention as disclosed herein. This assay can be used on any fish in principle, as long as the fish do not shoal. The preferred method uses zebrafish larvae however, due to their many advantages as an experimental vertebrate: small size, ease of care, cost (i.e. cheaper than rodents), genetic tractability, scalability (seen here in the simultaneous testing of many fish per experimental run), quick development time, short generation time, and fecundity.

The present invention is concerned in various aspects and embodiments with a method of screening for a substance or gene that affects activity or effect of a second gene, or activity or effect of a treatment, on behavior or physiology of a fish, the method comprising:

providing fish transgenic for the second gene or subject to said treatment, as model fish for screening;

mutating said model fish to provide mutated fish or treating said model fish with a test substance to provide treated fish;

comparing behavior or physiology of mutated fish or treated fish with model fish in order to identify any mutated fish or treated fish with altered behavior or physiology compared with model fish;

thereby to identify a test substance that affects activity or effect of the second gene or activity or effect of said treatment, or by identifying a genetic difference between model fish and mutated fish with such altered behavior or physiology to identify a gene that affects activity or effect of the second gene or activity or effect of said treatment.

The invention in various aspects and embodiments provides various modifications and developments of such a method.

Thus, for example, in one aspect the present invention provides a method of screening for a substance or gene (termed herein “first gene”) that affects activity or effect of a second gene, or activity or effect of a treatment, on a fish, the method comprising:

providing, as model fish for screening, (i) fish transgenic for the second gene, wherein the second gene is under regulatory control of a specific promoter and expression of the second gene within the fish affects an aspect of behavior or physiology of the fish, or (ii) fish subject to said treatment, wherein the treatment affects an aspect of behavior or physiology of the fish;

mutating said model fish to provide mutated fish or treating said model fish with a test substance to provide treated fish;

comparing an aspect of behavior or physiology of mutated fish or treated fish with that of model fish in order to identify any mutated fish or treated fish with altered behavior or physiology compared with model fish;

thereby to identify a test substance that affects activity or effect of the second gene or activity or effect of said treatment, or, by identifying a genetic difference between model fish and mutated fish with such altered behavior or physiology to identify a first gene that affects activity or effect of the second gene or activity or effect of said treatment.

Optionally, such a method further comprises screening for and preferably identifying or obtaining a chemical that interacts with the protein encoded by the wild-type first gene, e.g. for use as a therapeutic in the treatment of pain.

A specific promoter may be used, and a specific promoter is generally tissue-specific and/or inducible or derepressible.

A preferred promoter allows the disease state to be recapitulated, whilst also allowing all subsequent steps in the screening procedure to be carried out. Allowing expression of the disease in entirety under the control of its natural promoter, as described with previously disclosed prior art, may not permit these subsequent steps to be performed, and, in those circumstances in which they could be performed, they may not offer the equivalent ability to identify a therapeutic relevant to the treatment of human disease. An inducible promoter may be responsive to an applied stimulus, while a promoter that can be derepressed is active upon removal of a repressor. In some preferred aspects of the present invention the specific promoter may not be eye-specific in the fish and/or the behavior or physiology of the fish that is compared may not be vision, although in other aspects and embodiments eye-specific expression may be employed and/or assessment and comparison of vision. In various preferred embodiments of the invention, the specific promoter is selected from the group consisting of nicotinic acetylcholine receptor beta3, rhodopsin, Fli1, keratin8, islet-1, Type II cytokeratin, muscle creatine kinase, alpha actin, acidic ribosomal phosphoprotein P0, Beta actin, Pdx1, insulin, alpha1 tubulin, transducin, CRX, phosphodiesterase, ath5, brn3c, alphaB crystallin, tyrosine hydroxylase, dopamine decarboxylase, tyrosinase, GATA-2 and GATA-1 promoters. Tissues in which a harmful gene may be expressed include, but are not restricted to: neurons, subsets of neurons (including motor neurons), components of the visual system (e.g. photoreceptors, lens, ganglion cells), muscle, components of the auditory system, the skin, the swim bladder, the pancreas, the haematopoetic system (including specific haematopoetic subtypes), the vasculature and the heart.

Promoters which have already been shown to direct expression to specific cell types in zebrafish include:

Neuronal cells: nicotinic acetylcholine receptor beta3 (nAChRbeta3) promoter [Tokuoka, 2002]

Photoreceptors: rhodopsin promoter [Perkins, 2002].

Blood vessels: Fli1 promoter [Lawson, 2002].

Stratified epithelium: keratin8 promoter [Gong, 2002].

Motor neurons: islet-1 promoter [Higashijima, 2000].

Skin: Type II cytokeratin promoter [Ju, 1999].

Muscle: Muscle creatine kinase promoter [Ju, 1999]; Alpha actin promoter [Higashijima, 1997].

General expression: acidic ribosomal phosphoprotein P0 (arp) gene [Ju, 1999]; Beta actin promoter [Higashijima, 1997].

Pancreas: Pdx1 and insulin promoters [Milewski, 1998; Huang, 2001].

Neuronal progenitors: alpha1 tubulin promoter [Goldman, 2001]; GATA-2 promoter [Meng, 1997].

Haematopoetic cells: GATA-1 promoter [Long, 1997; Meng, 1999].

Lens: alphaB crystallin [Posner, 1999]

In preferred embodiments, the aspect of behavior or physiology that is to be determined for model and mutated and/or treated fish is gradable, i.e. can be quantitated.

The present invention provides in various aspects and embodiments for applying to or exposing fish such as zebrafish to two opposing stimuli. This allows for increased sensitivity and gradability of an assay and allows teasing apart of small differences in response tendency. Opposing stimuli may be selected for example from any combination of light stimulation, optomotor stimuli, temperature, whether with discrete changes or a temperature gradient, food, aversive chemicals or drugs, attractive or additive chemicals, physical aversion such as electric shock and a threatening shape.

In screening for analgesics, preferred embodiments employ a temperature gradient.

A temperature gradient may be maintained by a battery of underlying heaters and coolers which act to give a very stable gradient. Temperature parameters may be set by means of thermostats, if included in the apparatus.

At one end of the gradient, a temperature may be employed that is greater than normally tolerated or readily tolerated by fish. At one end of the gradient, a temperature may be employed that is less than normally tolerated or readily tolerated by fish. Where temperatures are too hot or too cold (e.g. greater than 28° C., e.g. 30, 35, 40 or 35-40° C., or less than 28° C., e.g. 24° C., respectively), fish will tend to stay away from those areas. Nociceptors respond to a minimum of 40° C. in the trout (Sneddon 2003). As with other screens involving application of stimuli that are unpleasant to the fish (e.g. electric shocks, a noxious chemical), a substance that provides an analgesic effect when applied to fish will tend to allow them to tolerate a worse stimulus, e.g. hotter or colder temperature, greater electric shock. Fish will then move into areas they previously avoided.

In embodiments of the invention, the fish (larval or adult) are introduced into long channels of water, along the length of which a temperature gradient is maintained, ranging from temperatures higher than those normally preferred by fish (40° C.) to those lower than normally preferred (24° C.). The location of the fish is tracked over time, starting from when they are introduced to the gradient. Analysis of the behavior of many fish in such a gradient will show whether they have an increased, decreased or normal sensitivity to temperature through analysis of their preferred location in the temperature gradient.

However, if an analgesic is effective, fish will not perceive a difference between an area in which an unpleasant stimulus is applied or is applied at an otherwise intolerable degree and other areas, so fish will randomly distribute between the areas. In order to provide enhanced discrimination between fish in which an analgesic effect is achieved, or is achieved to a greater degree than in others, a second stimulus may be applied with the aim of encouraging or forcing fish into areas into which they would not normally or readily go. Thus, for example, a stimulus may be applied that will drive fish into an area of excess heat, or severe cold, which they would normally avoid. The stimulus should not be so strong that it overrides the tendency to avoid the unpleasant stimulus, e.g. excess heat or cold, or electric shock. Fish should only be pushed into the previously intolerable area on application of the second stimulus when there is an analgesic effect. The second stimulus may for example be dark—graded filters may be applied to create areas of different degrees of shading or darkness. Zebrafish larvae do not like the dark and will tend to move towards lighter areas. A variation on this is moving the edge of an opaque piece of material over the fish towards the noxious stimulus. Fish perceive this as a predator moving over the water and swim into the light. They will not however tend to move into areas of excess heat or cold or electric shock, given a pain or nociception inducing stimulus, but with an analgesic effect they will be driven away from the darker areas towards the lighter areas. Another stimulus that can be used to move fish with reduced pain transduction or perception towards the painful stimulus is the optomotor response. A further option is to use fish that are addicted to a substance which draws them towards the substance when present in a particular region of the tank.

For example, zebrafish may be habituated to an additive substance, which may be nicotine. This may be through the addition of the substance to fish water, e.g. for a period of 3 days, before the water is then replaced with fresh water. The natural response is one of desire to receive more of the additive substance, e.g. further nicotine. The fish will now swim preferentially towards a focal source of the addictive substance, such as supplied through a wick to one end of the tank.

As noted, a graded threshold assay may involve passing a weak electric shock through the water. A particular strength of shock will cause pain to fish. Analgesics will increase the shock needed to cause a behavioral escape response. See Ehrensing at al, 1982, for proof of principle work on goldfish.

An assay in accordance with the invention may simply allow fish to choose a region within a gradient (e.g. of temperature or electric shock) without any other stimulus affecting them. Possible variations to this include addition of an opposing stimulus that would encourage fish to swim into an unpleasant region, e.g. hotter water. If such a stimulus is presented, and the level of that stimulus can be graded, there would be a level at which control fish are no longer willing to choose hot water and swim against the additional stimulus back into cooler water. Fish treated with analgesic would still be able to withstand the hotter water, and follow the additional stimulus into hotter water. This variation can be viewed as an enforced threshold response.

As noted, there are several opposing stimuli that will drive fish into the hotter water:

Optomotor response. This involves moving images, e.g. provided by placing a computer screen showing a movie of vertical stripes, which flow towards the hot end directly over the temperature gradient tank. The fish swim with the motion of the stripes. The sophistication of stimuli may be built up to allow a detailed, graded assessment of fish, e.g. zebrafish, visual function. Moreover, the assessment mechanisms allow for the testing of larger numbers of fish in a short period of time. A moving grating or a movie, e.g. presented as a computer-animated display on a screen, elicits innate optomotor behavior in zebrafish larvae; they swim in the direction of perceived motion (Orger et al. Nat Neurosci 2000 Nov. 3(11):1128-33). Zebrafish larvae innately begin responding to moving stimuli shortly after hatching. This is typically a very strong response. This stimulus may be graded (made weaker) by decreasing the contrast. Stimuli of different strengths may be employed to find the strength at which control fish will not enter water of a certain temperature whereas analgesic fish will. Hyperalgesic fish will have a lower threshold.

Light/dark stimulus. Larval fish show a strong preference for light conditions. Thus fish can be driven into hot water by covering the cool end of the gradient to make it dark. The stimulus may be graded by using materials of differing opacities. Again, a material can be found the opacity of which no longer forces control fish into hot water, but which analgesic treated fish are still capable of choosing light conditions. How much of the gradient is covered up (i.e. how close towards the hot end should the material extend) depends on the opacity of the material and how the fish have been treated.

Depth of water. Fish larvae prefer deep water to shallow water. The tank and gradient maintaining apparatus may be modified to accommodate a tank with a sloping bottom or sections of different depths, the shallow end being the cool end in either case. Analgesic treated fish should choose deep water over an increase in temperature whereas control fish should choose shallow water to avoid hotter water.

Fishes' behavior in a gradient may be constantly monitored by a video camera placed directly above the testing channels. It may be that fish can only be tested one per channel, and in which case, many thin channels may be employed in parallel. The data may be processed in many ways, including time spent in a certain area per fish, speed of motion, number of turns, mean location of a fish in the gradient, temperature range explored by individual fish. The motion parameter of choice may then be plotted as histograms for the two (or more) populations of fish tested. A difference in behavior will be reflected in a shift between the distributions. This shift may be analyzed statistically. Other statistical tests are of course possible with the data sets.

In addition to or instead of using a gradient, thermal nociception may be induced in a homogenous temperature. This may be done in a variety of ways, for example:

Place fish in a chamber, the water in which is within the temperature preference range, e.g. 28° C. Heat the water gently by placing the chamber containing the fish in a water bath which is being heated and stirred. Measure the temperature increase in the fish's chamber with a thermometer. Note when the fish starts to show signs of discomfort such as increased mobility, darting or thrashing. Take the fish through a series of different temperature chambers, noting again at what temperature the fish begins to show the signs of discomfort. Successive chambers contain water progressively hotter (e.g. by 1° C.) than the previous one, for example. Cooler temperatures than those normally preferred may also be tested.

In another approach, mechanical nociception may be employed. This may involve use of fine networks of hairs or mesh through which the fish must swim, driven by one of the above stimuli (heat gradient, OMR, light/dark, water depth). The fish are encouraged by the stimuli to swim though a series of increasingly tight structures that will deform their body wall more and more as they progress through them. They will reach a point at which they are no longer willing to swim through the structures in order to follow the stimulus. Fish which have increased or decreased mechanosensitive thresholds will progress further or less far through the series of structures respectively.

Chemical nociception may be employed, e.g. exploiting fishes' behavior when placed in solutions outside of their pH preference range (pH6-8). Discomfort behaviors involve increased mobility, darting, and thrashing. Fish are taken through a series of chambers containing solutions of increasing or decreasing pH either side of their pH temperature range (unbuffered). Fish may be contained in small sieves for ease of transfer between solutions. The behavior of the fish is noted in each solution. Fish treated with analgesics that block chemical nociception should show reduced discomfort behaviors.

All of these assays may be videoed to allow higher numbers of fish to be assayed, or allow re-evaluation of the data as some assays involve somewhat subjective measurements.

The sensitivity of the assessment of the degree of pain can also be increased by measuring a non-behavioral marker. For example, certain genes are differentially expressed in particular pain states, as described earlier. Further, neuronal activation can be assessed by measuring calcium fluxes. Measuring one of these non-behavioral markers provides further measurement in addition to the measurement of a purely behavioral output, thus increasing the scope and sensitivity of the pain assay.

By these means, together with a mutagenesis or chemical screen, genes and drugs may be identified which alter the transduction or perception of pain.

The present invention in certain aspects and embodiments provides for screening for and preferably identifying or obtaining a substance that provides a synergistic combination with another substance, or for screening for and preferably identifying or obtaining two or more substances that together provide a synergistic combination. Clinical benefit is often derived from synergistic combinations of drugs. Use of an in vivo system in accordance with the present invention allows for identification of such synergistic combinations.

Thus, in certain embodiments the invention comprises generation of a model fish, as disclosed, treating model fish with two or more substances, at least one of which is a test substance, and comparing the effect of the two or more substances in combination (whether simultaneously or sequentially applied) on an aspect of behavior or physiology on application of an unpleasant stimulus such as excess heat, excess cold or electric shock, with the effect of either or both of the two or more substances when applied individually or alone. Either all (or both) of the substances applied may each be a test substance, or one of the substances may be a drug known to have a beneficial effect on pain, or at least an effect in the model fish.

The invention thus provides for screening for and preferably identifying or obtaining a substance that provides an additive effect to a known drug or a synergistic effect with the known drug. It also provides for screening for and preferably identifying or obtaining a combination of two or more substances that provide a synergistic effect, compared with the effect of the two substances when employed individually or alone.

In addition to a test substance, the fish may be a mutated fish rather than a wild-type fish. It is then possible to assay for interacting effects, either beneficial synergistic effects, or deleterious effects, of the mutation plus the test substances. Alternatively, the analysis may be of a known therapeutic agent and the genetic mutation in order to discover either a new drug target of benefit in combination with the known drug, or a genetic marker of use in predicting which patients are most likely to benefit (or not benefit) from prescription of the known drug.

A diverse library of drug-like compounds, such as the LOPAC library (Sigma) may be used, or the Chembridge PHARMACOphore diverse combinatorial library. Other targeted libraries against particular targets classes may be used, such as ion channel libraries or G protein libraries.

Still further provided by the present invention is a method of identifying mutations, genotypes, allelic variations, haplotypes and genetic profiles associated with responsiveness to an analgesic. There is an increasing move towards targeted prescribing, whereby the choice of therapeutic is influenced by genotyping the patient. Particular polymorphisms have been found to predict both the therapeutic effectiveness of a compound, and also the likelihood of suffering certain side effects. Such rationalized prescribing is cost-effective. It also makes clinical trials easier to run, as likely responders can be targeted, thus necessitating a smaller sample size to achieve statistical significance. However, for the moment, most drugs, both already prescribed or in development, do not have an appropriate test.

The present invention provides for assessing the effectiveness of various medications in combination with random genetic mutations to identify those mutations which either enhance or decrease the therapeutic effectiveness and/or alter the side effect profile. This allows for identification of genes, polymorphisms, mutations, alleles and haplotypes associated with a particular response to an analgesic drug or other treatment, enabling development of appropriate genetic assays in humans to permit rationalized prescribing.

In a further embodiment, rather than target the prescribing of a beneficial agent, or improve the efficacy of an already beneficial agent, the invention may be used to reduce the side effects of an agent which otherwise might not be prescribed because of its negative side effect profile. In this situation the deleterious side effect is assayed, with an improvement of this deleterious side effect being examined for through the result of an additional chemical or interactor gene.

A method of the invention may comprise mutating model fish transgenic for the second gene to provide mutated fish and identifying a first gene that affects activity or effect of the second gene.

A method of the invention may comprise treating with a test substance model fish transgenic for the second gene to provide treated fish and identifying a test substance that affects activity or effect of the second gene.

A method of the invention may comprise mutating model fish subject to said treatment to provide mutated fish and identifying a first gene that affects activity or effect of said treatment.

A method of the invention may comprise treating with a test substance model fish subject to said treatment to provide treated fish and identifying a test substance that affects activity or effect of said treatment.

A method of the invention may comprise identifying a first gene that lessens activity or effect of the second gene.

The second gene may be known or hypothesized to be involved in pain transduction.

A method of the invention may comprise identifying a first gene that enhances or increases activity or effect of the second gene.

A method of the invention may comprise identifying a first gene that lessens activity or effect of the second gene.

A method of the invention may comprise identifying a test substance that lessens activity or effect of the second gene or said treatment.

A method of the invention may comprise identifying a test substance that enhances activity or effect of the second gene or said treatment.

In advantageous embodiments of the present invention, a fish model is generated by application of a chemical or physical treatment, rather than by genetic mutation, although use of genetic mutation is involved in other embodiments of the present invention as disclosed herein. Chemical or physical induction of an altered state allows all fish in a population or test sample to have the state induced at the same time in a controlled fashion, and then tested for genetic or chemical rescue. It can be used to overcome difficulties of disease lethality, and allows for rapid model generation. In preferred embodiments, the phenotype is gradable, which is advantageous in rescue screening.

The creation of a genetically altered line is time-consuming. Additionally, if the mutation affects survival or breeding capacity, maintenance of the genetically altered line may be compromised. Furthermore, if the mutation has early developmental effects, in addition to its disease-causing effects, this may compromise the appearance of the disease phenotype. Finally, when considering subsequent screening for rescue, as only a proportion of any clutch derived from breeding of the parent carriers will manifest the disease, it is necessary to either screen populations of fish rather than individual fish, or invent additional screening steps to allow the identification of the carrier fish (see elsewhere herein). This is because if a fish appears normal, one would not otherwise know whether this was because it was a wild-type, or a mutant fish which had been rescued from disease.

The induction of disease by chemical or physical methods can be used to overcome many of these issues. Chemicals are known to induce disease in mammals with phenotypic equivalence to human disease. For example, intraperitoneal injection of streptozotocin into mice induces pancreatic beta cell death after a period of 2 weeks [Hassan, 2001], phenotypically resembling type 1 diabetes mellitus. Chemicals have also been shown to cause specific defects in fish as part of a screen to dissect the genetics of development [Peterson, 2000]. It is by combining this disease induction with suitable phenotypic screening methods and rescue strategies (which may involve treatment with a test substance and/or mutation) that agents relevant to the rescue of human disease can be identified.

The present invention allows the assessment of an acute pain response. It is also highly desirable to be able to assay for a chronic pain response. The invention further provides methodology to allow this.

Chronic pain response has been demonstrated by the authors in this system by sensitizing fish with an irritant, DNCB. Sensitized fish locate to cooler areas of the temperature gradient than controls. Wholemount immunohistochemistry to detect levels of TNF-alpha confirmed that larvae raised in DNCB had mounted an inflammatory response whereas control larvae had not.

Nociception and pain may be induced by:

Induction of inflammation such as IBD or RA using pro-inflammatory agents such as TNBS or DSS.

Bathing fish in irritant solutions, such as dithranol, capsaicin, menthol, castor oil, ricinoleic acid, TPA (12-O-Tetradecanoyl phorbol-13-acetate), acids.

Injection of noxious substance into the muscle, brain ventricles or blood using microinjection equipment. Substances include capsaicin, acids, formalin, carrageenan, kainic acid, and lipopolysaccharides.

Irritation of the skin by thermal means, burning body surface with UV light, burning an area of the trunk skin with a hot needle.

Creation of a lesion/injury, cutting of the body surface, lesion of nerves, for example of the trunk periphery, crushing a body part/organ, for example a pectoral fin or the tail.

Fish may be treated with a substance in a number of ways, either as treatment to create a primary phenotype in which fish are affected in an aspect of behavior or physiology, or in treating fish with a test substance in the course of a screen for a test substance able to alter an effect of a primary treatment or mutation on a primary phenotype. Fish may be contacted with a test substance, it may be touched or rubbed on their surface or injected into them. A test substance may be added to water in which they are, or in the case of a protein, produced in the cell via expression of the appropriate coding sequence.

A different test substance may be added to each well of a multi-well plate, such as a 96 well plate, to identify that test substance exhibiting a beneficial or deleterious effect. There may be 1 or multiple fish in each well exposed to the test substance. The test substance may be added prior to the onset of the disease phenotype, concurrent with the onset of the disease phenotype, or subsequent to the onset of the disease phenotype. The same test substance may be added to different wells at different concentration. For example, test substance 1 may be added to well A1 at a concentration of 1 mM, to well A2 at a concentration of 100 uM, to well A3 at a concentration of 10 uM, to well A4 at a concentration of 1 uM and to well A5 at a concentration of 0.1 uM. Then test substance 2 to well B1 etc. The panel of test substances may be known drugs or new chemical entities.

Additionally, the test substances may be added in combination. For example, well A2 may contain test substance 1 and 2, well A3 test substance 1 and 3, well B2 test substance 2 and 3. Alternatively, every well may contain test substance x, with individual wells containing a panel of additional test substances.

In further preferred embodiments, the disease model in fish is generated by means of expression of a transgene that induces an effect on an aspect of behavior and/or physiology of the fish, a measurable and preferably gradable phenotype.

The promoter used to control expression, which may be tissue-specific expression may be inducible, which may facilitate establishment and/or screening of a fish line.

Nucleic acid may be manipulated in order to modify cells of fish such as zebrafish, as disclosed. Nucleic acid of a disease gene to be expressed in fish in accordance with the invention is to be integrated into the chromosome of cells. Integration may be promoted by inclusion of sequences which promote recombination with the genome, in accordance with techniques available in the art. The disease gene may be heterologous to the fish, e.g. may be heterologous to zebrafish (e.g. mammalian, such as human), and may be in wild-type form or in any allelic or mutant form. The disease gene may be a zebrafish or other fish gene, in wild-type or mutated form, e.g. to provide an extracopy of a zebrafish or other fish gene, such as in a mutated disease form.

Nucleic acid sequences encoding the peptides or polypeptides of the present invention may be readily prepared by the skilled person using the information and references contained herein and techniques known in the art (for example, see Sambrook and Russell “Molecular Cloning, A Laboratory Manual”, Third Edition, Cold Spring Harbor Laboratory Press, 2001, and Ausubel et al, Current Protocols in Molecular Biology, John Wiley and Sons, 1992, or later edition thereof). See Detrich et al. (1998) The Zebrafish: Biology. Methods in Cell Biology. Volume 59, and Detrich et al. (1998) The Zebrafish: Genetics and Genomics. Methods in Cell Biology. Volume 60 for techniques of zebrafish maintenance, mutagenesis, transgenesis and mapping.

The desired coding sequence may be incorporated in a construct having one or more control sequences operably linked to the nucleic acid to control its expression. Appropriate regulatory sequences, including promoter sequences, terminator fragments, polyadenylation sequences, enhancer sequences, marker genes and other sequences as appropriate may be included.

Regions responsible for promoter and enhancer activity of a gene known to be expressed in a desirable pattern such as only under certain conditions or in certain tissue, may be isolated by ligating stretches of sequence from upstream of the translation start codon in the gene to a reporter gene. Constructs with deletions in putative promoter and/or enhancer regions are generated and the constructs tested for tissue specific gene expression in transgenic fish, e.g. transgenic zebrafish, fugu, goldfish, medaka and giant rerio.

A selectable marker, for example gene encoding a fluorescent protein such as Green Fluorescent Protein (GFP) may be included to facilitate selection of clones in which the gene construct has inserted into the genome. Where a fluorescent marker is used, embryos may be screened under a fluorescent dissecting microscope. Embryos, or fish into which they grow, may be screened for the presence of a defect resulting from the transgene. In another approach, embryos may be pooled prior to extraction of genomic DNA and analysis of the genomic DNA by PCR and/or restriction enzyme digest. Positive clones may be expanded and developed into breeding fish. These fish may then be bred to produce fish which carry one copy of the gene construct in the germ line.

These heterozygous fish may then be bred to produce fish carrying the gene homozygously.

In order to introduce a disease gene into a fish embryo, e.g. a zebrafish embryo, a gene construct is made, using techniques available to those skilled in the art. The construct may be released from a vector by restriction digest, and gel purified, for example by elution in 1×TE (pH8.0) and dilution to a working concentration of 50-100 ug/ml KCl containing a marker dye such as tetramethyl-rhodamine dextran (0.125%). Typically, 1 to 3 nl of this solution may be injected into single celled zebrafish embryos. Several thousand embryos may be injected.

Injected embryos are grown up and then mated with each other or to a non-transgenic wild-type fish. Transmission of the transgene to the subsequent generation is usually mosaic, ranging from 2 to 90%. At least 100 offspring are typically analyzed to establish whether the founder fish carriers the transgene.

Families from which fish with the appropriate characteristics came may be maintained through subsequent generations. This maintenance then allows this new mutant strain to be entered into a secondary screen in accordance with further aspects of the invention.

Another aspect of the present invention provides cells of transgenic fish, such as zebrafish, fugu, goldfish, medaka and giant rerio as disclosed, whether isolated cells or cell lines derived from the fish and optionally immortalized using standard techniques.

A gene such as a disease gene sequence (e.g. heterologous to fish, such as heterologous to zebrafish) to be employed in aspects and embodiments of the present invention may employ a wild-type gene or a mutant, variant or derivative sequence may be employed. The sequence may differ from wild-type by a change which is one or more of addition, insertion, deletion and substitution of one or more nucleotides of the sequence shown. Changes to a nucleotide sequence may result in an amino acid change at the protein level, or not, as determined by the genetic code.

It is well known that pharmaceutical research leading to the identification of a new drug may involve the screening of very large numbers of candidate substances, both before and even after a lead compound has been found. This is one factor which makes pharmaceutical research very expensive and time-consuming. Means for assisting in the screening process can have considerable commercial importance and utility. Such means for screening for substances potentially useful in treating or preventing a disorder or disease is provided by fish such as zebrafish according to the present invention. Suppressor genes identified using the invention and substances that affect activity of such suppressor genes represent an advance in the fight against disease since they provide basis for design and investigation of therapeutics for in vivo use, as do test substances able to affect activity or effect of a treatment, and substances that affect activity or effect of expression of a disease gene in a fish.

In various further aspects the present invention relates to screening and assay methods and means, and substances identified thereby.

The present inventors have realized that fish such as zebrafish are useful in a secondary suppressor or enhancer screen. A secondary suppressor screen involves introducing one or more mutations into the genome and screening or selecting for negation or suppression or enhancement of the effect of a primary mutation.

The principle can be illustrated by way of example, with reference to a hypothetical gene B of which normal function is to control fish size. If this gene is mutated so that it is underactive (a hypomorphic mutant), smaller fish will ensue. Now take another hypothetical gene S whose normal function is to make fish smaller. If this gene is mutated, such that the gene is also underactive, the fish will be bigger. Thus if a mutation is introduced into the S gene in a fish already harboring a mutated B gene, the two will cancel each other out and the fish will be normal sized. The mutated S gene suppresses the phenotype of the mutated B gene.

However, there is a problem where gene B is a dominant disease gene, since if this makes the fish non-viable, causes them to die quickly or fail to reproduce, it will not be possible to raise adult fish harboring a mutation in gene B.

The present invention provides a solution to this problem either by inducing the disease state by non-genetic means, or by restricting expression of the disease gene to one or more tissues or particular conditions, by placing it under the control of a suitable promoter, e.g. tissue specific and/or inducible, or by phenocopying the effect of the mutation at a defined time point. As a result of this spatially and/or temporally restricted expression, the disease process is limited to these specific cells. The fish are viable, can be raised to adulthood and bred, and are thus amenable to use in a secondary suppressor screen.

Furthermore, the invention provides for an accurate, gradable and rapid screening method for the presence of a pain response, and its degree of transduction or perception. Use of a graded response is highly desirable for identifying a drug, as described earlier, and can be contrasted with phenotypic assays which measure an all or nothing response, such as the occurrence, or non-occurrence of seizures.

The fish, e.g. zebrafish, provided by the present invention are useful in screens for interactor, e.g. suppressor, genes that affect activity or effect of a second gene in the fish, such as a disease gene. According to a further aspect of the present invention there is provided the use of a fish, e.g. zebrafish, fugu, goldfish, medaka and giant rerio in such a screen.

As noted, model fish may be generated using chemical and/or physical means, in which case the invention also provides for screening for a gene that has an effect on the aspect of behavior of physiology that is affected by the chemical or physical treatment.

Thus, the aspects of the invention involve genetic rescue of an induced phenotype.

Zebrafish are particularly amenable to genetic rescue experiments.

Mutagens such as ethylnitrosourea (ENU) may be used to generate mutated lines for screening e.g. screening, in either the F1-3 (for dominant) or F3 (for recessive) generations. (It is only by the third generation that recessive mutations can be bred to homozygosity.) ENU introduces point mutations with high efficiency, so any phenotype is most likely to be recessive. Retroviral vectors may be used for mutagenesis, and although they are an order of magnitude less effective than ENU they offer the advantage of rapid cloning of a mutated gene (see e.g. Golling et al. (2002) Nat Genet 31, 135-40. Mariner/Tc family transposable elements have been successfully mobilized in the zebrafish genome and may be used as mutagenic agents (Raz et al. (1998) Curr Biol 8, 82-8. ENU remains the most efficient and easy method available at the moment, and so is preferred for now.

The mapping of mutant genes is comparatively easy. The density of markers on the fish genetic map is already considerably greater than that of the mouse map, despite the relatively recent popularity of zebrafish. Consult the harvard website on zebrafish, findable using any available web browser using terms “zebrafish” AND “harvard”, currently (28 Nov. 2002) found at (http://zebrafish.mgh.harvard.edu/mapping/ssr_map_index.html), The Sanger Centre has begun to sequence the zebrafish genome with sequence currently (28 Nov. 2002) published at www.ensembl.org/Danio_rerio/. The site can be found using any web browser using terms “danio rerio” and “Sanger” or “ENSEMBL”. Around 70,000 ESTs have been identified and are being mapped on a radiation-hybrid map.

Another strategy for introducing effects, which may be random, on an aspect of behavior or physiology in accordance with the present invention, is to down-regulate the function or activity of a gene, for instance employing a gene silencing or antisense technique, such as RNA interference or morpholinos. A gene found using the invention to be involved in pain transduction or perception may be downregulated using such techniques. These can be either targeted against candidate genes, or generated against an array of genes as part of a systematic screen. It is relatively easy to inject RNA, DNA, chemicals, morpholinos or fluorescent markers into fish embryos, including zebrafish embryos given their ex utero development.

A morpholino is a modified oligonucleotide containing A, C, G or T linked to a morpholine ring which protects against degradation and enhances stability. Antisense morpholinos bind to and inactivate RNAs and seem to work particularly well in zebrafish. Some disadvantages with this approach include the a priori need to know the gene sequence, the need to inject the chemical into the early embryo, potential toxic side effects and the relatively short duration of action. Additionally, they knock down the function of a gene, and thus do not offer the same repertoire of allele alterations as point mutations.

A further strategy for altering the function of a gene or protein as part of an in vivo screen, coupled to any of the various other components of the screening strategy disclosed herein, is to generate transgenic lines expressing protein aptamers, crossing these with the disease lines, or inducing disease by other means, then assaying for an altered disease state. Protein aptamers provide another route for drug discovery [Colas, 1996] but the ability to assay their effectiveness in vivo in accordance with the present invention markedly increasing their usefulness beyond in vitro screening methods.

In a further aspect, the present invention provides a method of screening for a suppressor gene that lessens activity or effect of a disease state, the method comprising:
providing fish, e.g. zebrafish transgenic for a disease gene under regulatory control of a promoter, wherein expression of the disease gene within cells or tissue of the fish affects an aspect of behavior or physiology of the fish, as model fish for screening;
subjecting said model fish to mutation to provide mutated fish;
comparing behavior or physiology of mutated fish with behavior or physiology of model fish in order to identify any mutated fish with altered behavior or physiology compared with model fish;
identifying a genetic difference between model fish and any mutated fish with such altered behavior or physiology, thereby to identify a suppressor gene that lessens activity or effect of the disease gene.

As noted, preferred embodiments of the present invention in its various aspects employ zebrafish.

Of course, the person skilled in the art will design any appropriate control experiments with which to compare results obtained in test assays.

A number of strategies are available to the ordinary skilled person for altering gene expression, including the use of morpholinos, RNAi and Pnas, or through introducing a secondary mutation into a disease state fish.

Mutagenesis may be performed as follows:

Ethylnitrosourea (ENU) is dissolved in acetic acid to a final concentration of 10 mM, as determined by the optical density at 238 nm at pH6.0 (extinction coefficient=5830/M/cm), and then diluted to a working concentration of 3.0 mM in 10 mM sodium phosphate buffer, pH 6.6. Males which reliably produce fertilized offspring are placed in ENU solution for 1 hour. After the procedure the fish are washed in 2 changes of aquarium water for 1 hour each time, prior to return to the aquarium. The mutagenesis procedure is repeated up to 6 times at weekly intervals.

The frequency of mutations induced is proportional to the exact number of mutagenesis procedures performed. The number of procedures can thus be varied depending on the number of mutations desired per genome.

The actual mutagenesis procedure is best carried out in the dark to minimize the stress to the fish.

Initial progeny from the mutagenized fish are mosaic. The mutagenized fish are therefore mated 3 times at weekly intervals following the final mutagenesis procedure. Progeny obtained after this will be non-mosaic, since any mutations will have arisen in spermatogonial stem cells.

Other useful mutagenesis agents include gamma- or X-ray-mediated mutagenesis, and retrovirus-mediated insertional mutagenesis.

Following identification of a gene which affects activity or effect of a second gene or disease state, e.g. a suppressor gene, the gene (including a homologue in another species, e.g. human) or encoded gene product may be cloned or otherwise provided in an isolated or purified form, and may be provided in a composition comprising at least one additional component.

Often there will already be enough confidence in similarities in biological pathways to move straight to human or another mammal. However, certain steps may help.

Where there is a mutated gene leading to rescue, the human homologue of that gene may be introduced into the rescue line in both wild-type and mutated form. If the human gene has equivalent action in its mutated form, then rescue will be seen when it is injected in the mutated form, but may be lost when injected in the wild-type form, depending on the mechanism of action of the mutated gene.

Where drugs are already known to act against the rescuing gene or its encoded protein, these can be screened directly. As this is easy to do because of the attributes engineered into the system, as disclosed herein, this is quicker to do than embarking on an exploration of the equivalence of biological pathways.

Where only possible drugs are known that act against related proteins to the rescuing encoded protein, then these can all be screened. Again, the scalability of the system described above makes this a cost-effective way to proceed.

Where the rescuing protein proves to be a poor target, or where a rescuing protein remains elusive, gene and protein microarrays and gene and protein profiling techniques may be used to identify potential targets. These approaches can generate many false leads and conventionally require much work to identify real lead candidates. However, using the present invention, the effort required to screen candidate drugs or chemicals against dozens or hundreds of possible targets is less than that required to further validate these individual targets.

As noted, the gene, e.g. suppressor gene, (including a homologue in another species, e.g. human) or a gene product encoded by the gene, e.g. suppressor gene, may be used in a screening system for assaying ability of a test substance to affect activity of the gene or the gene product encoded by the gene.

A test substance that affects activity of a gene, e.g. a suppressor gene, or the gene product encoded by the gene may be provided in a composition comprising at least one additional component.

Following identification of a suppressor gene for a disease gene of interest, or other gene that affects activity or effect of a second gene, the suppressor or other gene and/or an encoded gene product may be employed as a target for identification of potential therapeutics or as a therapeutic in its own right. Also, the nature of the suppressing or other effect may be investigated further.

The suppressor or other gene that affects activity or effect of a second gene may be a novel gene or may be a known gene not previously known to have a function of affecting or suppressing activity or effect of the relevant disease gene. The gene may be one already known or suspected to have function in affecting or suppressing activity, in which case the results from the fish assay add weight to the available evidence. In particular, the fact that the suppression or other effect occurs in vivo increases the confidence for using the gene, or encoded gene product or fragment thereof, or a component in the pathway of action of the gene or gene product, as a drug target. For further investigation and use, a homologue from another species may be used, where available e.g. via use of cloning or screening technology.

The responsible mutation, e.g. suppressive mutation, may be identified by using mapping techniques available in the art, (e.g. see Detrich H. W., Zon L. I. & Westerfield M. (1998) The Zebrafish: Genetics and Genomics. Methods in Cell Biology. Volume 60, pg 182-192).

Thus, to identify the position of the relevant mutation, e.g. a suppressive mutation, the mutant locus is mapped relative to the position of a marker, the position of which is known. DNA markers include short sequences of DNA, cloned genes or other mutations. The current best method in zebrafish involves simple sequence length polymorphisms (SSLPs) as they cover the entire genome at high density. It is therefore possible to map to within 0.5 cM, from which either a chromosomal walk may be initiated, further mapping may be undertaken using single strand conformational polymorphisms, or candidate genes selected directly.

Mapping Using SSLP

These markers consist of 2 primers flanking a dinucleotide (CA) repeat. These are extremely variable in length & polymorphic between zebrafish strains. The SSLP mapping involves the following steps:

Raising a map cross, identifying mutant carriers, fixing mutant & sibling progeny separately

Isolating genomic DNA from both mutants & siblings

Genome scanning using pooled DNA from both mutants & siblings to determine linkage group

Verifying potential linkages with single embryo DNA

Searching for closely linked markers

Positioning the mutation on the genetic map by determining the number of recombinations between marker & mutation.

Isolation of Genomic DNA

To extract DNA from single embryos, embryos fixed in 100% methanol are poured into a petri dish. More methanol is added to the dish to ensure the embryos remain covered. Embryos are then pipetted into a 96 well plate: a single embryo per well. A pipette is then used to remove as much methanol as possible from around the embryos. The remaining methanol is then evaporated off on a PCR block set at 70° C. for 15 minutes. 25 ul of a mix of 250 ul proteinase K (17 mg/ml, Merck) & 2.25 ml 1×TE, is added to each well. The PCR plate is then covered with Hybaid film & heated in a PCR machine for 240 minutes at 55° C., followed by a 10 minute 75° C. incubation to inactivate the proteinase K. The plates can be kept at −20° C. until needed.

Genome Scanning

Pooled DNA is prepared by taking 10 ul from each of 48 single samples, and then diluted to a final concentration of 50 ng/ul. Primers for markers are arranged on a master primer 96 well plate in such a way that the mutant & sibling sample analyzed with the same marker will subsequently run adjacent to each other on an agarose gel. The markers selected for the PCR plates are those known to show useful polymorphisms & which evenly span the entire genome.

PCR reactions are then set up in 96 well format. Each well contains 14.28 ul PCR mix, 0.16 ul each of 20 uM forward & reverse primer, 0.4 ul of 5 U/ul Taq polymerase & 5.0 ul of template DNA. PCR is performed with initial denaturing at 94° C. for 3 minutes, followed by 35 cycles of denaturing at 94° C. for 30 seconds, annealing at 60° C. for 30 seconds & primary extension at 72° C. for 1 minute. The reaction is completed by a final 5 minute extension at 72° C.

PCR Mix

0.2 mM dATP
0.2 mM dCTP
0.2 mM dGTP
0.2 mM dTTP

in PCR buffer

PCR Buffer (10×)

100 mM Tris-HCl, pH 8.3
500 mM KCl
15 mM MgCl₂
0.1% (w/v) gelatin
   Single-Embryo PCR

PCR reactions are set up and performed as above, except that single embryo DNA is used as the template.

The PCR products are assessed for polymorphisms by running out on a 2% agarose gel at 200V for 80 minutes in 1×TBE.

Mapping Using SSCP

This uses single strand DNA. Each strand assumes its thermodynamically preferred conformation. Single nucleotide substitutions may alter the conformation sufficiently for a difference in migration pattern to be detected on a non-denaturing gel. This allows non-SSLP markers tightly linked to the mutation to be analyzed.

The protocol used to amplify a marker is as for SSLP mapping. To precipitate the PCR products, 2 volumes of pre-cooled 100% ethanol and 0.1 volume of 3M Na-acetate are added to the PCR product, vortexed well, incubated for at least 20 minutes at −20° C. & centrifuged in a cooled centrifuge at 13000 rpm for 25 minutes. The supernatant is discarded, the DNA pellet air-dried & resuspended in 8 ul of ddH₂O. To 5.4 ul of PCR product, 0.6 ul of denaturing solution & 2.4 ul of loading buffer are added & briefly mixed, prior to incubation at 85° C. for 10 minutes. The sample is then quickly chilled on ice. 6-8 ul of each sample is then loaded onto a native precast acrylamide gel (CleanGel SSCP, ETC Elektrophorese-Technik) & run at 200V & 15° C. following the manufacturer's instructions.

Denaturing Solution

10 mM EDTA
500 mM NaOH
   Loading Buffer
2% bromophenol blue
2% xylenecyanol
in formamide

The gels are then stained using a silver staining kit (PlusOne DNA Silver Staining Kit, Pharmacia), as per the manufacturer's instructions.

By these methods the mutation is mapped close enough to select a candidate gene. This gene is then sequenced in both mutant wild-type fish to identify mutations.

If the suppressor or other gene that affects activity or effect of a second gene encodes a protein, it may be that that protein interacts with or binds the second gene, e.g. disease gene., or gene product. Thus, for example, a novel protein-protein binding pair may be identified, immediately presenting the possibility of modulating or affecting such binding as a target for identifying candidate therapeutics.

Where interaction or binding between gene products is to be investigated further or employed in assay methods for identifying further substances able to affect the binding or interaction, suitable approaches are available in the art, for instance techniques involving radioimmunoassay, co-immunoprecipitation, scintillation proximity assay, ELISA methods, and two-hybrid assays (see e.g. Fields and Song, 1989, Nature 340; 245-246), for instance using the two binding domains of the GAL4 transcription factor or the LexA/VP60 system.

Further mutation in the suppressor or other gene may be used to identify variants with enhanced or otherwise altered suppressor function.

Thus, the suppressor gene or other gene, or encoded gene product, in wild-type or a mutated form (which may be a mutated form as identified in the original screen or a further mutated form) may be used in a therapeutic composition.

In various further aspects, the present invention thus provides a pharmaceutical composition, medicament, drug or other composition comprising a suppressor gene or other gene or gene product or substance found to affect the disease gene of interest or suppression of the disease gene of interest, the use of such a material in a method of medical treatment, a method comprising administration of such a material to a patient, e.g. for treatment(which may include preventative treatment) of a medical condition, use of such a material in the manufacture of a composition, medicament or drug for administration for such a purpose, e.g. for treatment of a proliferative disorder, and a method of making a pharmaceutical composition comprising admixing such a material with a pharmaceutically acceptable excipient, vehicle or carrier, and optionally other ingredients.

One or more small molecules may be preferred therapeutics identified or obtained by means of the present invention. However, the invention may be used to identify appropriate targets for antibody mediated therapy, therapy mediated through gene targeting or protein targeting, or any of a variety of gene silencing techniques, including RNAi, antisense and morpholinos.

Whatever the material used in a method of medical treatment of the present invention, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

Pharmaceutical compositions according to the present invention, and for use in accordance with the present invention, may include, in addition to active ingredient, a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required.

Examples of techniques and protocols mentioned above can be found in Remington's Pharmaceutical Sciences; 16th edition, Osol, A. (ed), 1980.

Vectors such as viral vectors have been used in the prior art to introduce nucleic acid into a wide variety of different target cells. Typically the vectors are exposed to the target cells so that transfection can take place in a sufficient proportion of the cells to provide a useful therapeutic or prophylactic effect from the expression of the desired peptide. The transfected nucleic acid may be permanently incorporated into the genome of each of the targeted cells, providing long lasting effect, or alternatively the treatment may have to be repeated periodically.

A variety of vectors, both viral vectors and plasmid vectors, are known in the art, see U.S. Pat. No. 5,252,479 and WO 93/07282. In particular, a number of viruses have been used as gene transfer vectors, including papovaviruses, such as SV40, vaccinia virus, herpesviruses, including HSV and EBV, and retroviruses. Many gene therapy protocols in the prior art have used disabled murine retroviruses.

As an alternative to the use of viral vectors in gene therapy other known methods of introducing nucleic acid into cells includes mechanical techniques such as microinjection, transfer mediated by liposomes and receptor-mediated DNA transfer, also administration of naked DNA or RNA, by simple administration, e.g. injection, of nucleic acid such as a plasmid, for instance to muscle.

All documents mentioned anywhere in this specification are incorporated by reference.

The invention is now further illustrated in worked embodiments providing technical support. Further aspects and embodiments of the present invention will be apparent to those skilled in the art in the light of the disclosure herein.

### EXAMPLE 1

1. The ambient environment was prepared through the use of an air handling system to maintain a constant air temperature below 24° C., enabling maintenance of a stable water gradient.

2. A shallow tray was taken of dimensions 2 cm deep, 30 cm long and 20 cm wide. This was filled with oxygenated water from the aquarium, the composition of which is suitable sustain zebrafish. This water was cooled to less than the lowest temperature desired in the gradient.

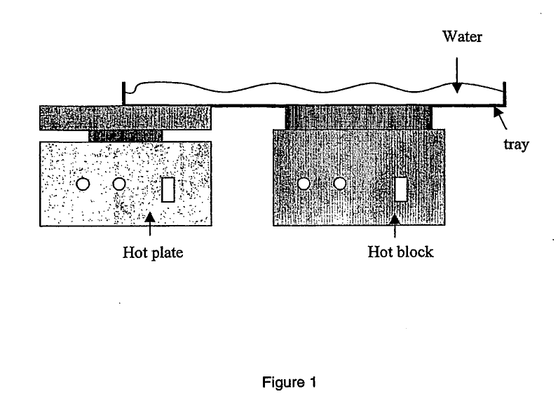

3. The tray was placed on top of two heating plates: a hot plate, capable of heating to high temperatures, and a hot block. The heights of the blocks were adjusted so that when the tray is placed on top of them the tray lies flat (FIG. 1). It was important to ensure the heating surfaces were symmetrically located under the long axis of the tray to ensure equal heating of both longitudinal halves of the tray.

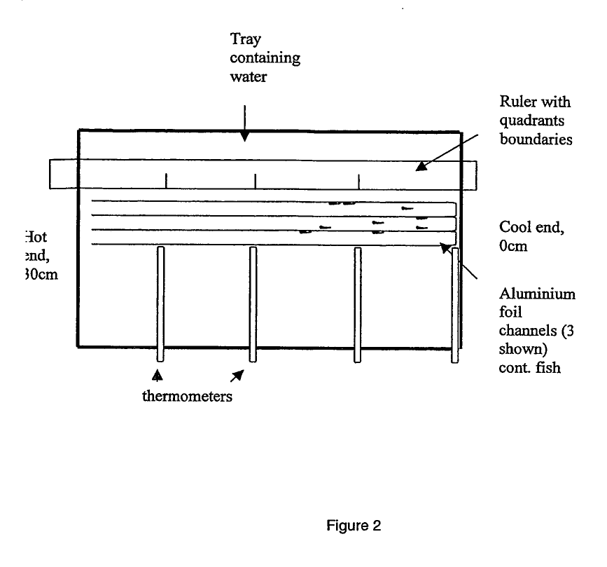

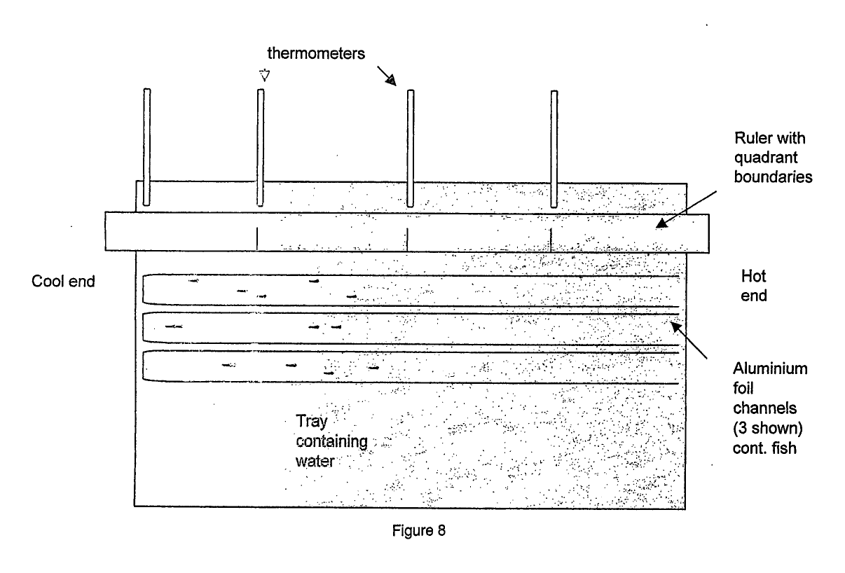

4. A transparent 30 cm ruler was placed on top of the tray and the boundaries of 4 quadrants marked on it: 0-7.5 cm, 7.5-15 cm, 15-22.5 cm and 22.5-30 cm (FIGS. 2 and 8). These serve to split the gradient up into areas as a means of summarizing temperature choice.

5. Aluminum foil was used to make channels for the fish to swim in. These were water tight and closed at one end and open at the other. The channels should be deep enough so that water does not over flow from the channel to the rest of the tray when the channels are touching the bottom. The open end is necessary so that as water evaporates from the hot end the channels don't dry up. They were then placed in the water with the closed end at the cool end. They were then filled with water. The channels were in the middle of the tray, i.e. not closer to one long side of the tray than the other.

6. The hotplate and hotblock were turned on to achieve a temperature of about 40° C. maintained at the hot end, with the unheated end reaching only 24° C. To measure the temperature at each quadrant boundary, thermometers were placed near the channels at each boundary.

7. When a stable gradient was achieved, fish were taken directly from the fish room (28° C.). As much of the liquid around them as possible was removed. They were sucked up with a 1.5 ml Pasteur pipette and placed in the water where the temperature was 28° C. The idea is to introduce all fish to be tested to the channels at the same time and with minimal perturbation of the temperature gradient. When testing more than one channel of fish, the others should be introduced as quickly as possible. Multiple channels, each with multiple fish, may be tested at one time.

8. The experiment was timed with a stopwatch. The experiment should run for 10 minutes in the first instance as this is typically how long it will take a fish to explore its environment and choose an area. Note the temperatures at the quadrant boundaries at t=0 mins.

9. At 2, 5 and 10 minutes, the number of fish in each quadrant was counted. A ruler was placed over the channel to facilitate location of the quadrant boundaries.

10. At the end of the 10 minutes, the fish were removed as gently as possible with a pipette. The temperatures were also rechecked to ensure that the gradient had been stable.

The data were then processed using a statistical software package. Fish treated with an analgesic spend more time at higher temperatures than wild-type fish, which quickly withdraw from the noxious stimulus. Fish that experience hyperalgesia showed a lower threshold to temperature and spend less time than controls in warmer temperatures.

Results

To show that this assay can detect changes in a larval zebrafish's ability to sense thermal stimuli, a group of fish whose nociceptive senses have been experimentally changed had to be shown to be different to untreated control fish.

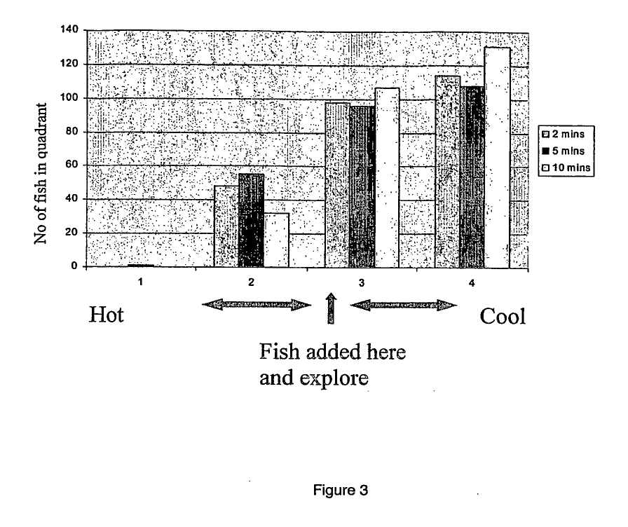

FIG. 3 shows control data. Wild-type untreated fish (less than 10 days old) take 10 minutes to choose cooler water. n=260. The x axis shows the quadrants, 1 being hot water and 4 the coolest. Fish are added at t=0 mins as indicated. By 10 minutes, there was a shift in the population to cooler temperatures.

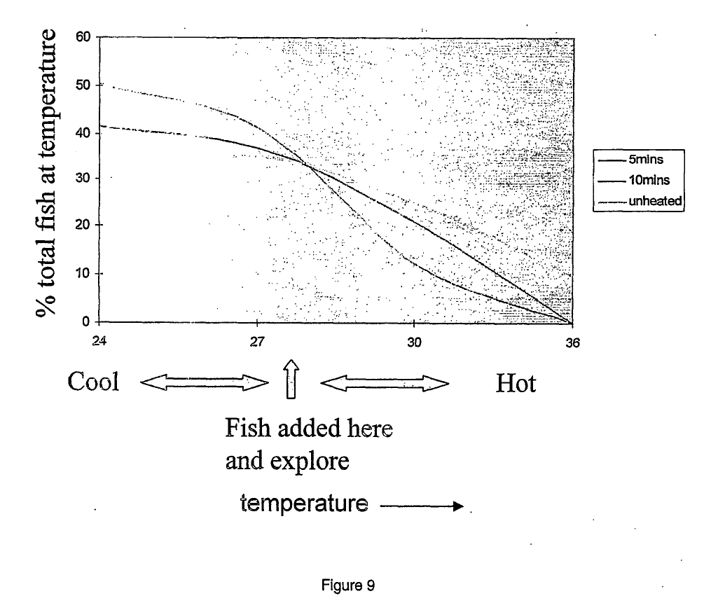

FIG. 9 shows the same data represented as a line graph with the x axis showing temperature in ° C.

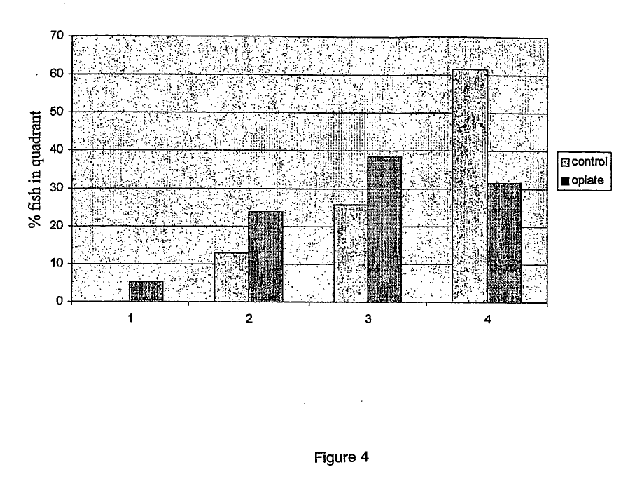

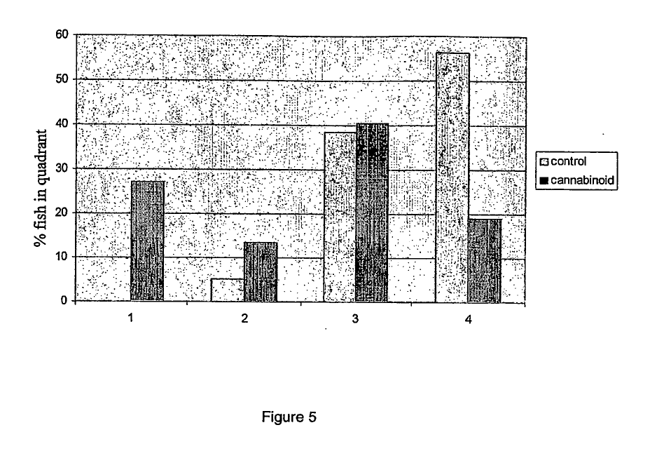

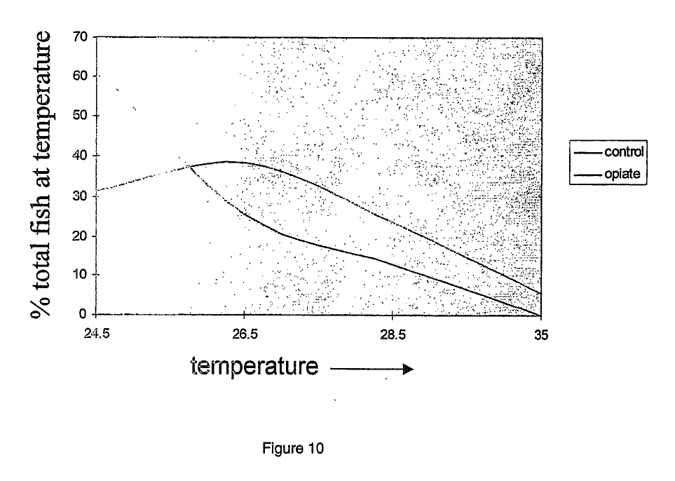

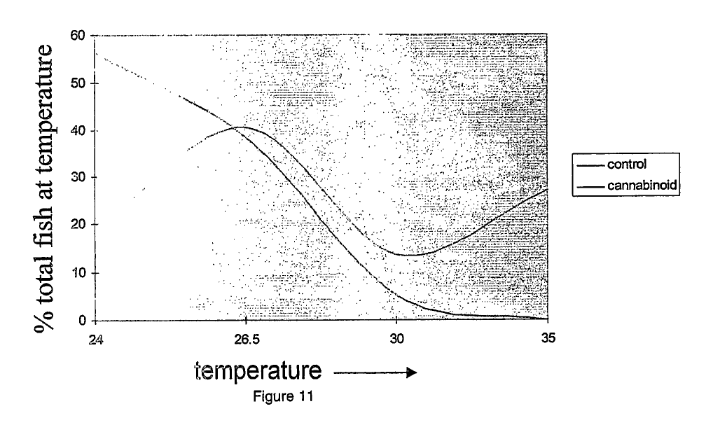

FIGS. 4 and 5 show analgesic data from experiments using fish at less than 10 days old. FIGS. 10 and 11, respectively, show the same data represented as line graphs with the x axes showing temperature in ° C.

Batches of 20 fish were dosed with either an opiate (FIGS. 4 and 10) or a cannabinoid (FIGS. 5 and 11) and left at 28° C. for 30 minutes before testing.

Details of the analgesics used:

Opiate: GR 89696 Fumerate. 1 ug/ml in DMSO. Stock solution of 2 mg/ml in DMSO, kept AT −20° C.

Cannabinoid: Anandamide (an endogenous cannabinoid). 5 ug/ml. Stock 1 mg/ml in ethanol, stored at −20° C.

Dosing: place 20 larvae in a well of a 24-well cell culture plate. Remove as much liquid from around the fish as possible. Add 2 ml of embryo medium plus 1 ul of GR 89696 Fumerate stock or 10 ul of anandamide stock. Mix by swirling the liquid. Leave fish at 28C for 30 mins. Remove as much liquid as possible and then suck up all fish in one go if possible and place gently in the temperature gradient.

### EXAMPLE 2

Induction and Rescue of Hyperalgesia as Caused By Immersal in DNCB

Hyperalgesia was induced by placing the whole animal in an irritant solution to cause inflammation of the body surface, such as 1 ug/ml dinitrochlorobenzene. This was made up from a 2 mg/ml stock in ethanol, stored in the dark at 4° C. After dosing, the fish were placed at 28° C. for 2 h, then tested.

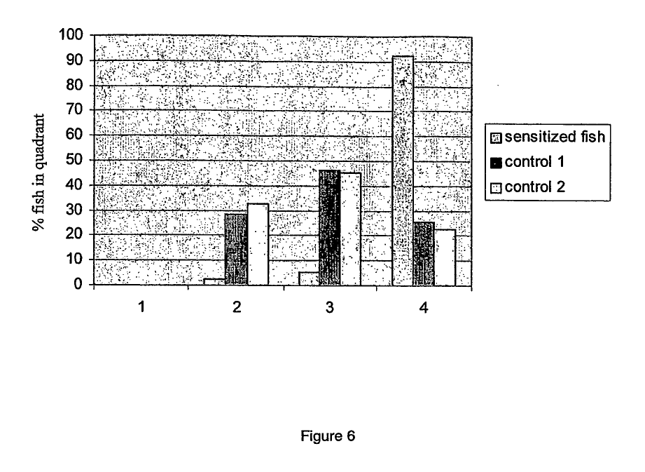

FIG. 6 shows results indicating that fish (less than 10 days old) sensitized with DNCB choose cooler temperatures very strongly and quickly as they over-react to the temperature. t=5 mins.

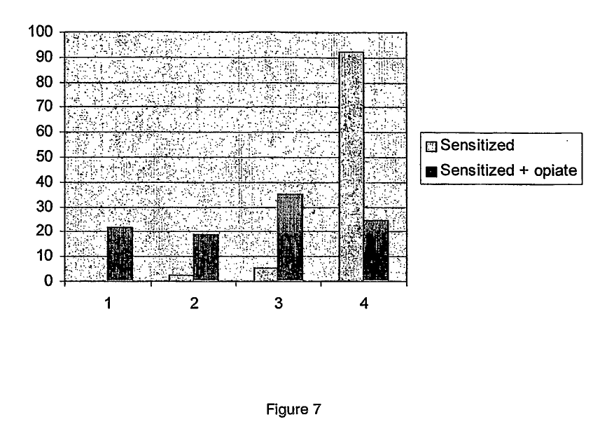

FIG. 7 juxtaposes the same data from sensitized fish from FIG. 6 with the same fish treated with an opiate and retested. t=5 mins.

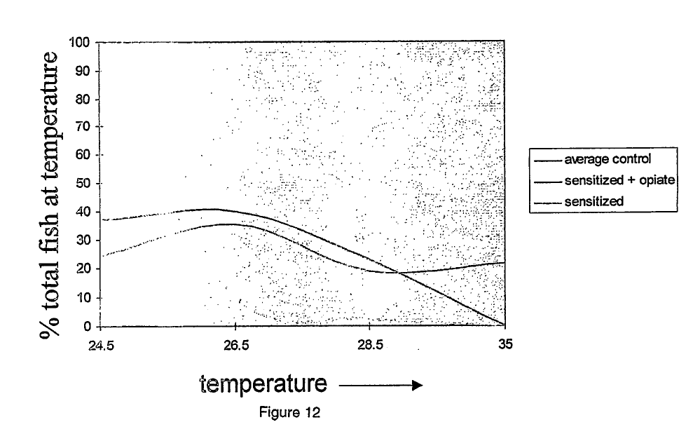

FIG. 12 is a line graph representation combining the data shown in FIGS. 6 and 7 the x axis showing temperature in ° C. FIG. 12 shows results that demonstrate that fish sensitized with DNCB locate to cooler temperatures than unsensitized controls and do so very rapidly. When these same fish are treated with an opiate and retested they lose this sensitivity, and their distribution becomes randomized in the gradient. The controls for these 2 tests are shown for comparison. Assay performed at t=5 mins.

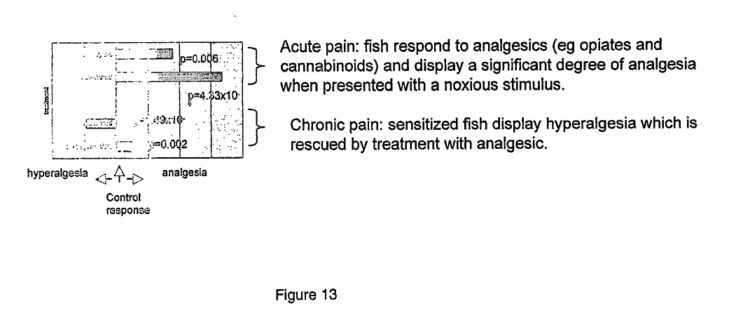

FIG. 13 indicates the degree of hyperalgesia or analgesia (that is, length and direction of histogram bars with respect to the control response). This is derived from the difference in distribution of the fish in the channels between the treated group and the control group for that experiment. The p values are calculated using t tests.

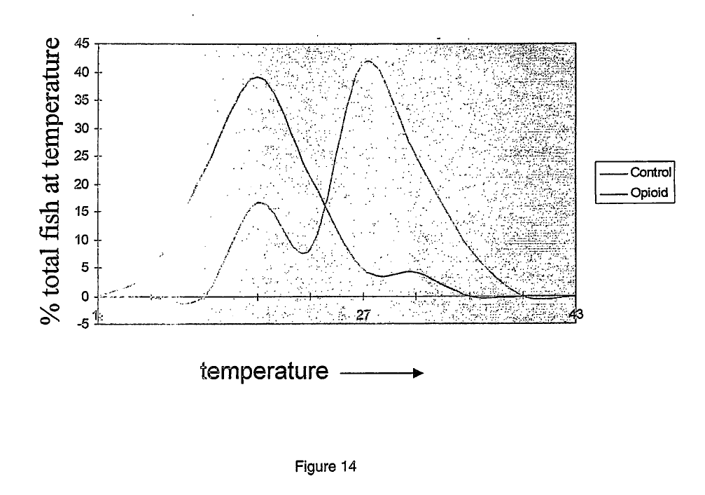

FIG. 14 shows results indicating that three week old fish respond in essentially the same way as younger fish (all other data shown in other figures are obtained using fish less than 10 days old) when treated with an opiate. Opiate treated fish locate to regions of a higher temperature than untreated fish. The x axis shows temperature in ° C.

### REFERENCES

Adamska, et al. (2000) Mech Dev 97, 161-5.
Ashworth, R. and Bolsover, S. R. (2002). Brain Res Dev Brain Res (139): 131-137.
Bang et al. (2002) J Neurosci Methods 118, 177-87.
Brand et al. (1994) Methods Cell Biol 44, 635-54.
Brand and Perrimon (1993) Development 118, 401-15.
Calissendorff (1976) Acta Ophthalmol (Copenh) 54, 109-17.
Colas et al. (1996) Nature 380, 548-50.
Creton, R., Speksnijder, J. E. and Jaffe, L. F. (1998). J Cell Sci (111):1613-1622.
D'Avino and Thummel (1999) Methods Enzymol 306, 129-42.
Driever et al. (1996) Development 123, 37-46.
Duffy (2002) Genesis 34, 1-15.
Eells (2000) Neurotoxicology 21, 321-30.
Fetcho, J. R., Cox, K. J. and O'Malley, D. M. (1998). Histochem J (30): 153-167.
Furukawa et al. (2002) J Neurosci 22, 1640-7.
Ghalie et al. (2002) Neurology 59, 909-13.
Goldman et al. (2001) Transgenic Res 10, 21-33.
Gong et al. (2002) Dev Dyn 223, 204-15.
Haffter et al. (1996) Development 123, 1-36.
Hassan and Janjua (2001) J Ayub Med Coll Abbottabad 13, 26-30.
Heng et al. (1999) Invest Ophthalmol Vis Sci 40, 190-6.
Higashijima et al. (2000) J Neurosci 20, 206-18.
Higashijima et al. (1997) Dev Biol 192, 289-99.
Higashijima, S., Masino, M. A., Mandel, G. and Fetcho, J. R. (2003). J Neurophysiol (90): 3986-3997.
Huang et al. (2001) Mol Cell Endocrinol 177, 117-24.
Ju et al. (1999) Dev Genet 25, 158-67.
Kelley et al. (1995) Development 121, 3777-85.
Kennedy et al. (2001) J Biol Chem 276, 14037-43.
Kerr, R. Lev-Ram, V., Baird, G., Vincent, P., Tsein, R. Y. and Shafer, W. R. (2000). Neuron (26): 583-594.
Lawson and Weinstein (2002) Dev Biol 248, 307-18.
Link et al. (2001) Dev Biol 236, 436-53.
Long et al. (1997) Development 124, 4105-11.
Long et al. (2000) Mech Dev 97, 183-6.
Meng et al. (1997) Proc Natl Acad Sci USA 94, 6267-72.
Meng et al. (1999) Blood 93, 500-8.
Milewski et al. (1998) Endocrinology 139, 1440-9.
Nicolson et al. (1998) Neuron 20, 271-83.
Odenthal et al. (1996) Development 123, 391-8.
Perkins et al. (2002) Vis Neurosci 19, 257-64.
Peterson et al. (2000) Proc Natl Acad Sci USA 97, 12965-9.
Posner et al. (1999) Biochim Biophys Acta 1447, 271-7.
Saszik et al. (1999) Vis Neurosci 16, 881-8.
Scheer et al. (2002) Mech Dev 112, 9-14.
Solnica-Krezel et al. (1994) Genetics 136, 1401-20.
Taurog (1976) Endocrinology 98, 1031-46.
Tokuoka et al. (2002) J Neurosci 22, 10324-32.
van de Wyngaert et al. (2001) Acta Neurol Belg 101, 210-6.
Vihtelic and Hyde (2000) J Neurobiol 44, 289-307.
Webb, S. E. and Miller, A. L. (2003). Zygote (11): 175-182.
Wyttenbach et al. (2000) Proc Natl Acad Sci USA 97, 2898-903.
Yeh and Hsu (2000) (Danio rerio). Biosci Biotechnol Biochem 64, 592-5.
Zimprich, F., Ashworth, R. and Bolsover, S. (1998). Pflugers Arch (436):489-493.
Bermond, B 1997 The myth of animal suffering. In: Dol, M, Kasanmoentalib, S, Lijmbach, S, Rivas, E and van den Bos, R, Animal Consciousness and Animal Ethics. Van Gorcum Publishers, The Netherlands.
Beukema, J. J. (1970) Neth. J. Zool., 20: 81-92.
Domanik et al. Arch Environ Contam Toxicol. 1978;7(2):193-206.
Ehrensing et al. (1982) Carassius auratus. Pharmacology, Biochemistry and Behavior, 17: 757-761.
Miller and Ogilvie Bull Environ Contam Toxicol. 1975 November;14(5):545-51
Sneddon et al. Proc R Soc Lond B Biol Sci. 2003 Jun. 7;270(1520):1115-21.
Sneddon LU Brain Res. 2003 May 16;972(1-2):44-52.
Rose, J. D. A critique of the paper by Sneddon et al. ‘Do fish have nociceptors: Evidence for the evolution of a vertebrate sensory system’.
http://uwadmnweb.uwyo.edu/Zoology/faculty/rose/Critique%20of %20Sneddon%20article.pdf
Verheigen and Buwalda (1988) Do pain and fear make a hooked carp in play suffer? CIP-GEGEVENS. Utrecht. ISBN 90-9002167-1.
Welch et al. Bull Environ Contam Toxicol. 1989 November;43(5) :761-8

1. A method of screening for a first gene or substance that affects a disease state associated with pain or nociception, or a pain state or nociception, the method comprising:
providing fish with a disease state associated with pain or nociception, or a pain or nociception state, as model fish for screening;
mutating said model fish to provide mutated fish or treating said model fish with a test substance to provide treated fish;
comparing the pain state or nociception of mutated fish or treated fish with the pain state or nociception of model fish in order to identify any mutated fish or treated fish with altered pain state or nociception compared with model fish;
thereby to identify a test substance that affects a disease state associated with pain or nociception, or a pain state or nociception, or, by identifying a genetic difference between model fish and mutated fish with such altered pain state or nociception, to identify a gene that affects a disease state associated with pain or nociception, or a pain state or nociception.

2. A method of screening for a substance or first gene that affects activity or effect of a second gene, or activity or effect of a treatment, on a fish, the method comprising:
providing, as model fish for screening, (i) fish transgenic for the second gene, wherein expression of the second gene provides fish with a disease state associated with pain or nociception, or a pain or nociception state, or (ii) fish subject to said treatment, wherein the treatment provides fish with a disease state associated with pain or nociception, or a pain or nociception state;
mutating said model fish to provide mutated fish or treating said model fish with a test substance to provide treated fish;
comparing the pain state or nociception of mutated fish or treated fish with the pain state or nociception of model fish in order to identify any mutated fish or treated fish with altered pain state or nociception compared with model fish;
thereby to identify a test substance that affects activity or effect of the second gene or activity or effect of said treatment, or, by identifying a genetic difference between model fish and mutated fish with such altered pain state or nociception, to identify a first gene that affects activity or effect of the second gene or activity or effect of said treatment.

3. The method according to claim 2, comprising mutating model fish transgenic for the second gene to provide mutated fish and identifying a first gene that affects activity or effect of the second gene.

4. The method according to claim 2, comprising treating with a test substance model fish transgenic for the second gene to provide treated fish and identifying a test substance that affects activity or effect of the second gene.

5. The method according to claim 2, comprising mutating model fish subject to said treatment to provide mutated fish and identifying a first gene that affects activity or effect of said treatment.

6. The method according to claim 2, comprising treating with a test substance model fish subject to said treatment to provide treated fish and identifying a test substance that affects activity or effect of said treatment.

7. The method according to claim 1, wherein the test substance lessens activity or effect of the second gene or said treatment.

8. The method according to claim 1, wherein the test substance enhances activity or effect of the second gene or said treatment.

9. The method according to claim 1, wherein the first gene lessens activity or effect of the second gene.

10. The method according to claim 1, wherein the first gene enhances activity or effect of the second gene.

11. The method according to claim 1, wherein the first gene or test substance has an analgesic effect.

12. The method according to claim 2, wherein the second gene is involved in pain transduction.

13. The method of claim 1, further comprising screening for a further test substance that interacts with the protein encoded by the first gene or the test substance.

14. The method according to claim 13, further comprising identifying and/or obtaining the further test substance.

15. The method according to claim 13, wherein the interaction has a synergistic or additive effect.

16. The method according to claim 13, wherein the interaction between the test substance and the protein encoded by the first gene has a deleterious effect.

17. The method according to claim 1, wherein the pain state or nociception is assessed by the measurement of a behavior.

18. The method according to claim 17, wherein the behavior is the relative position of fish in a gradient induced by one or more stimuli.

19. The method according to claim 18, wherein when more than one stimulus is used, the stimuli are applied sequentially.

20. The method according to claim 18, wherein a stimulus is gradable such that it can be quantitated.

21. The method according to claim 20, wherein a stimulus is gradable by the use of opposing stimuli.

22. The method of claim 18, wherein one or more stimuli are selected from the group consisting of light stimuli, optomotor stimuli, temperature, food, aversive chemicals or drugs, attractive or additive chemicals or drugs, physical aversion stimuli such as electric shock or threatening shape, mechanical stimuli and depth of water.

23. The method according to claim 17, wherein the pain state or nociception is further assessed by the measurement of the expression of one or more genes.

24. The method according to claim 17, wherein the pain state or nociception is further assessed by the measurement of calcium fluxes.

25. The method according to claim 1, wherein the fish is selected from the group consisting of zebrafish, fugu, goldfish medaka and giant rerio.

26. The method according to claim 24, wherein the fish is a zebrafish.

27. A fish model for a disease state associated with pain or nociception, or a pain state or nociception, for screening for a substance or first gene that affects activity or effect of a second gene, or activity or effect of a treatment, on a fish, comprising (i) a fish transgenic for the second gene, wherein expression of the second gene induces a disease state associated with pain or nociception, or a pain or nociception state, or (ii) a fish subject to the treatment, wherein the treatment induces a disease state associated with pain or nociception, or a pain or nociception state.

28. The fish model according to claim 27, wherein the second gene is involved in pain transduction.

29. The fish model according to claim 27, wherein the pain state or nociception of the fish is assessed by the measurement of behavior.

30. The fish model according to claim 29, wherein the behavior is the relative position of fish in a gradient induced by one or more stimuli.

31. The fish model according to claim 27, wherein the fish is selected from the group consisting of zebrafish, fugu, goldfish medaka and giant rerio.

32. The fish model according to claim 31, wherein the fish is a zebrafish.
